(19) United States
(12) Patent Application Publication
(10) Pub. No.: US 2017/0273772 A1
(43) Pub. Date: Sep. 28, 2017

(54) Assemblies And Methodologies For The Repair Of Hernias

(71) Applicant: Yale University, New Haven, CT (US)

(72) Inventor: Kurt E. Roberts, Hamden, CT (US)

(21) Appl. No.: 15/504,841

(22) PCT Filed: Aug. 20, 2015
(86) PCT No.: PCT/US15/46041

§ 371 Date: Feb. 17, 2017

Related U.S. Application Data
(60) Provisional application No. 62/039,486, filed on Aug. 20, 2014.

(51) Int. Cl. A61F 2/00; A61B 17/068; A61B 17/04

(52) U.S. Cl. CPC A61F 2/0063; A61B 17/0401; A61B 17/068; A61B 2017/00663

(57) ABSTRACT

An assembly for the repair of a hernia. The assembly includes an elongated shaft having a first end; a first holding member at least mostly positionable in the elongated shaft and a second holding member positionable in the elongated shaft; a coupler assembly having (i) a coupler for coupling the first holding member to the second holding member and (ii) a locking arrangement; a deployment assembly for deploying at least one of the first and second holding members out of the elongated shaft; a restrainer for restraining a portion of the coupler prior to the deployment of the second holding member out of the first end of the elongated shaft; wherein the coupler can be tensioned so as to pull the first holding member and the second holding member towards each other and the locking arrangement is lockable about the coupler. Methods of repairing a hernia by closing a hole in the fascial layer of an abdominal wall are also disclosed.

This application claims the benefit of and priority to provisional application Ser. No. 62/039,486, filed Aug. 20, 2014, the subject matter of which is also incorporated by reference in its entirety.

### BACKGROUND OF THE INVENTION

The present invention is generally directed to assemblies and methodologies for closing an abdominal wall defect during laparoscopic/minimally invasive hernia repair with an easy abdominal wall defect closing system. The advantages include, but are not limited to, a facilitation of closure of the abdominal wall defect and the ability to achieve the results faster than traditional closing techniques.

Currently most defects are not closed primarily because it is technically too difficult and time consuming. Hence most abdominal wall hernias are closed by bridging the defect using a mesh patch. An improved and suitable method using a mesh patch is disclosed in copending international application No. PCT/US2014/038076, the subject matter of which is incorporated by reference as if fully set forth herein. However, it can also be desirable to close the abdominal wall defect first and then in addition, secure it with a mesh if desired. The two main advantages of this procedure include, but are not limited to, restoration of the functional abdominal wall and decreasing the chance of recurrence of the hernia. Using state of the art devices and methodologies, the recurrence rates are estimated to be 3-10% for traditional bridging the defect laparoscopic hernia repair technique with mesh.

Currently the abdominal wall defects are not getting closed in the vast majority of laparoscopic hernia repairs. In some cases surgeons elect to close the defect in addition to the bridging the defect with a mesh. In the cases where the defects are getting closed, surgeons use standard suturing techniques. Some surgeons use the costly robotic system to apply these traditional suturing techniques, which adds a huge cost to these surgeries.

Among other things, the present invention utilizes an improved holding rod assembly and technique that deploys two (2) holding rods to approximate the fascial edges of a hernia defect that are secured with a locking arrangement, such as, but not limited to, a self-locking knot.

### SUMMARY AND OBJECTIVES OF THE INVENTION

It is thus an objective of the present invention to provide more effective, more reliable, efficient, secure and safer repairs of hernias.

Still further it is an objective of the present invention to reduce the risk of a wound infection and subsequent infection during the repair of hernias.

Still further it is an objective of the present invention to save hospital procedure or operation time because of the ease of use of the present invention and performance of the methodologies disclosed herein.

Yet another objective of the present invention is to provide methodologies and assemblies for carrying out the methodologies that yields fewer complications like bleeding and chronic pain than achievable with prior art assemblies and methodologies.

Still further, another objective of the present invention is to facilitate the ability of basic laparoscopic surgeons to perform the improved methodologies as disclosed herein.

Moreover, another objective of the present invention is to provide for an increase in patient satisfaction for all the reasons noted herein.

Still other objects and advantages of the invention will in part be obvious and will in part be apparent from the specification.

The invention accordingly comprises the features of construction, combination of elements, sequence of steps and arrangement of parts which will be exemplified in the construction and methodology hereinafter set forth, and the scope of the invention will be indicated in the claims.

Therefore, and generally speaking, in accordance with a first embodiment, the present invention is directed to an assembly for the repair of a hernia, comprising an elongated shaft having a first end; a first holding member at least mostly positionable in the elongated shaft and a second holding member positionable in the elongated shaft; a coupler assembly comprising (i) a coupler for coupling the first holding member to the second holding member and (ii) a locking arrangement; a deployment assembly for deploying at least one of the first and second holding members out of the elongated shaft; a restrainer for restraining a portion of the coupler prior to the elongated shaft; a restrainer for restraining a portion of the coupler prior to the deployment of the second holding member out of the first end of the elongated shaft; wherein the coupler can be tensioned so as to pull the first holding member and the second holding member towards each other and the locking arrangement is lockable about the coupler.

In accordance with another preferred embodiment, a method of repairing a hernia by closing a hole in the fascia layer of an abdominal wall is provided. Using the assemblies disclosed herein, the method may comprise the steps of extending the first end of the elongated shaft through at least a fascia layer of the abdominal wall at a first position; deploying the first holding member out the first end of the elongated shaft; removing the elongated shaft out of the abdominal wall; and while the coupler is still coupled intermediate the first and second holding rods, extending the first end of the elongated shaft through at least the fascia layer at a second position; deploying the second holding member out the first end of the elongated shaft and removing the restraint upon the coupler; removing the elongated shaft out of the abdominal wall; applying a tension on the coupler to cause the first holding member and the second holding member to be pulled towards each other; locking the locking arrangement about the intermediate portion of the coupler, whereby a defect in the fascia layer is closed thereby.

In a specific embodiment, the holding members may be deployed while in the fatty layer of the abdominal wall. Alternatively, the holding members may be deployed simply “above” the fascia layer, as exemplified in FIG. 13.

### BRIEF DESCRIPTION OF THE DRAWINGS

For a fuller understanding of the invention, reference is had to the following description taken in connection with the accompanying drawings, in which:

FIG. 1 is a perspective view in part, cross-sectional view in part and plan view in part of an assembly constructed in accordance with a first embodiment of the present invention;

FIG. 2 is a view of the assembly of FIG. 1 after the first holding member has been deployed, in accordance with the present invention;

FIG. 3 illustrates the assembly of FIG. 1 showing how the restraining member is still in position across the opening until the second holding member is thereafter deployed out of the shaft 12, the latter of which is show in FIG. 4;

FIG. 4 illustrates the assembly of FIG. 1 after the second holding member has been deployed showing how the restraining member has been moved away from the opening after the second holding member has been deployed out of the shaft 12;

FIG. 5 (e.g. FIGS. 5A-5D) illustrates various deployment assembly embodiments and other features that may be used in connection with the present invention and preferred embodiments herein;

FIGS. 6A, 6B illustrate yet other preferred embodiments of the invention, in which FIG. 6A illustrates an assembly in which a different shaped opening is provided and the means for restraining is differently shaped than that shown in the first embodiment and FIG. 6B illustrates yet another preferred embodiment, in which a different restraining device is utilized, namely, gluing, taping and/or otherwise adhering a portion of the coupler to the outer surface of the elongated shaft either directly to the shaft or to something therebetween as would be understood in the art;

FIG. 7 illustrates yet another preferred embodiment, in which a notch or slot is provided at the top edge of the first end of the shaft 12. Here too, the shape of the notch, along with the use of a restrainer as disclosed herein, protects the coupler from being cut along the top edge 12B of the end 12A of the shaft while the holding members are being deployed;

FIG. 8-12 illustrate a sequence of preferred steps in carrying out a first embodiment using any one of the assemblies that may be constructed as set forth herein. Specifically, FIG. 8 is a view of the assembly of FIG. 1 having been inserted through layers of the abdominal wall and prior to deployment of the first holding member; FIG. 9 illustrates the assembly of FIG. 1 after the first holding member has been deployed and the assembly of FIG. 1 has been retracted out of the abdominal wall and the assembly has been moved into position on the other side of the hole in the abdominal wall for insertion therein, further illustrating how the coupler remains connected to the first and second holding members as the assembly has been moved into position on the other side of the hole in the abdominal wall for insertion; FIG. 10 illustrates the assembly of FIG. 1 having been inserted into the abdominal wall at the second position of the respective peritoneum and fascia layers; FIG. 11 illustrates the assembly of FIG. 1 after the second holding member has been deployed and the assembly has been retracted out of the abdominal layer and the first and second holding members are in position; and FIG. 12 illustrates the application of tension on the coupler pulling the first holding member and the second holding member towards each other thereby pulling the fascia layer at the first position towards the fascia layer at the second position towards each other, and the locking arrangement if lockable about the coupler; whereby a defect in the fascia layer is closed thereby.

FIG. 13 is similar to the embodiment of FIGS. 8-12 except that the first and second holding members are not deployed in the fatty layer of the abdominal wall. Rather, the first end will again pierce through the peritoneum layer in the defect as shown in FIG. 13. This process is repeated for the second holding member as would be understood by those skilled in the art after reviewing FIG. 13. In this way, and similar to the aforementioned embodiment, a tension on the coupler pulls the first holding member and the second holding member towards each other and, after the locking arrangement is locked about the coupler; a defect in the fascia layer is closed thereby.

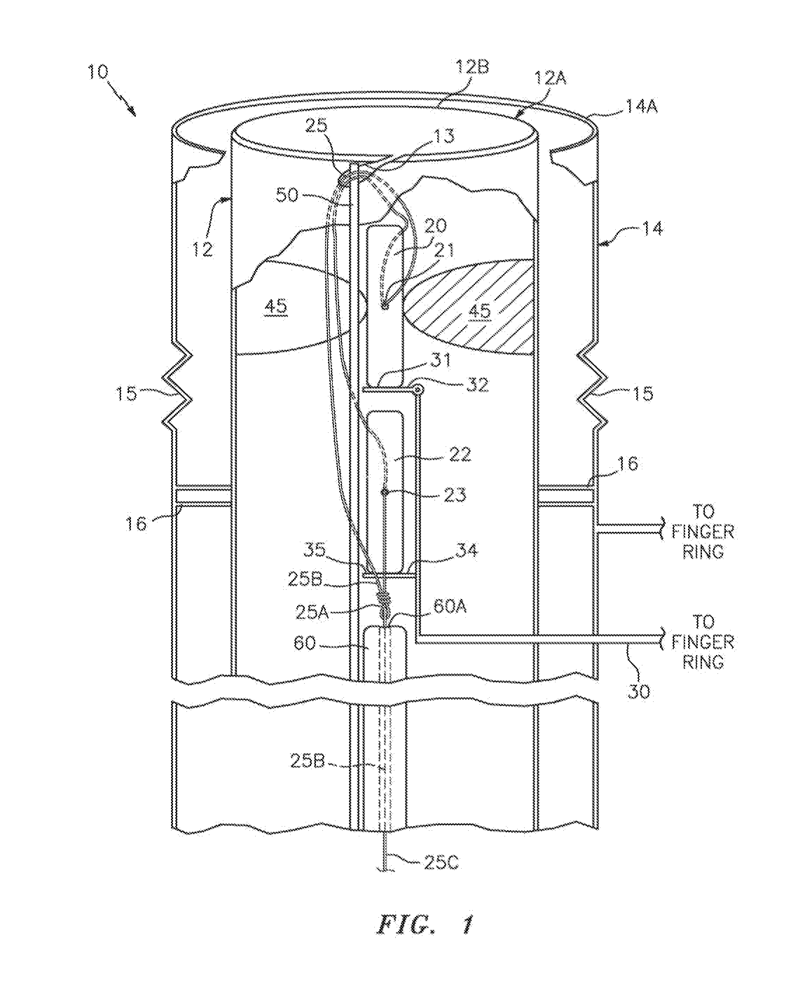

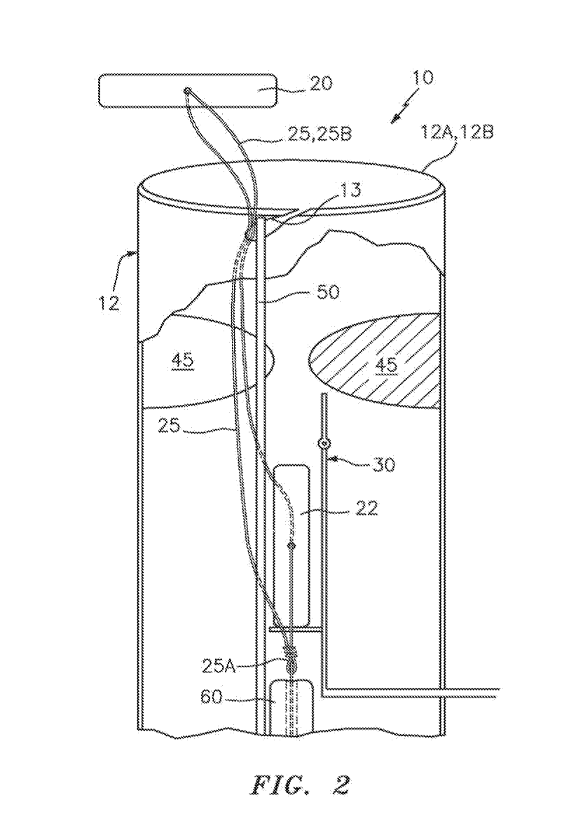

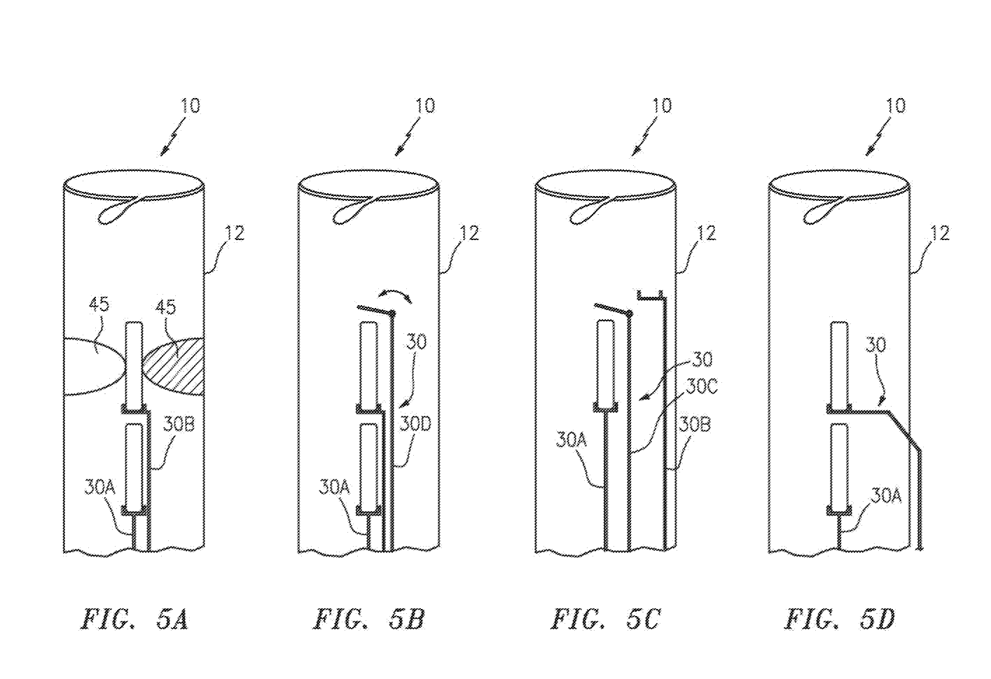

In addition, it will be recognized after a review of the current specification, that the figures are not all in scale among themselves or therebetween. For example, and not limitation, it can be seen that the holding members change in size/scale between the figures. Moreover, if each feature was illustrated to scale within each figure, there would be a need for larger paper or smaller images. Therefore, for the convenience of the reader, the scale has been adjusted for ease of understanding. Specific dimensions for components are provided where appropriate and/or helpful. Where not provided, it is readily assumed that those skilled in the art will understand the features and relative sizes disclosed herein. For example, it will be understood by those skilled in the art that holding members 20, 22 preferably do not simply “float” inside shaft 12. To the contrary, one or more protrusions or a ring may be provided within the inner shaft to retain the holding members 20, 22 accordingly, such as that illustrated in FIGS. 1 and 5A, although not shown to scale, but which should be understood by those skilled in the art. Lastly, like numbers to identify like parts and features will be used among the various figures, but not all features will be specifically identified in each illustration.

### DETAILED DESCRIPTION OF THE PREFERRED EMBODIMENTS

Generally speaking, to enter the abdominal cavity in accordance with preferred embodiments of the present invention, a standard laparoscopic port used during the laparoscopic hernia repair may be used. The device may be inserted thru a port and is advanced to one fascial edge of the hernia defect. Approximately 0.5 to 2 cm distance from the edge of the hernia defect the penetration portion of the device (hollow needle) is advanced thru the fascia. Once the opening of the hollow needle is above the fascia the first holding member may be deployed (e.g. pushed) out and would be thus located above the fascia. Then the hollow needle is pulled back below the fascia. A suture is coupled to this first holding member as well as coupled to a second holding member that would still be located in the assembly. The assembly is then moved to the opposing edge on the contralateral side of the hernia defect. The hollow needle is advanced thru the fascia in approximately 0.5 cm to 2 cm distance from the edge of the hernia defect. Once the opening of the hollow needle is above the fascia, the second holding member is pushed out and is located above the fascia. The hollow needle is then pulled back below the fascia. Bothe holding members are now deployed above the fascia and are connected by the suture. This suture is formed in a loop, which means it preferably goes thru both holding members. A self-tightening knot may be provided in connection with the suture. The suture would then be hanging out the very end of the assembly, i.g. through the laparoscopic part outside the patients' body. A knot pusher (e.g. shaft 60) may also be provided and positioned such that is can be advanced through the assembly and outside the front tip of the assembly but still inside the abdominal cavity. Now, by applying tension on the suture outside the body and simultaneously advancing the knot pusher that advances the self-tightening knot, the loop of the suture is closing and both holding members will be pulled towards each other, which accomplishes a closing of the gap of the hernia defect. The final step once the locking arrangement (e.g. self-tightening knot) is in place (i.e. meaning the fascial edges may be touching each other and the hernia defect is closed in that area), the suture may be severed between the knot and the assembly.

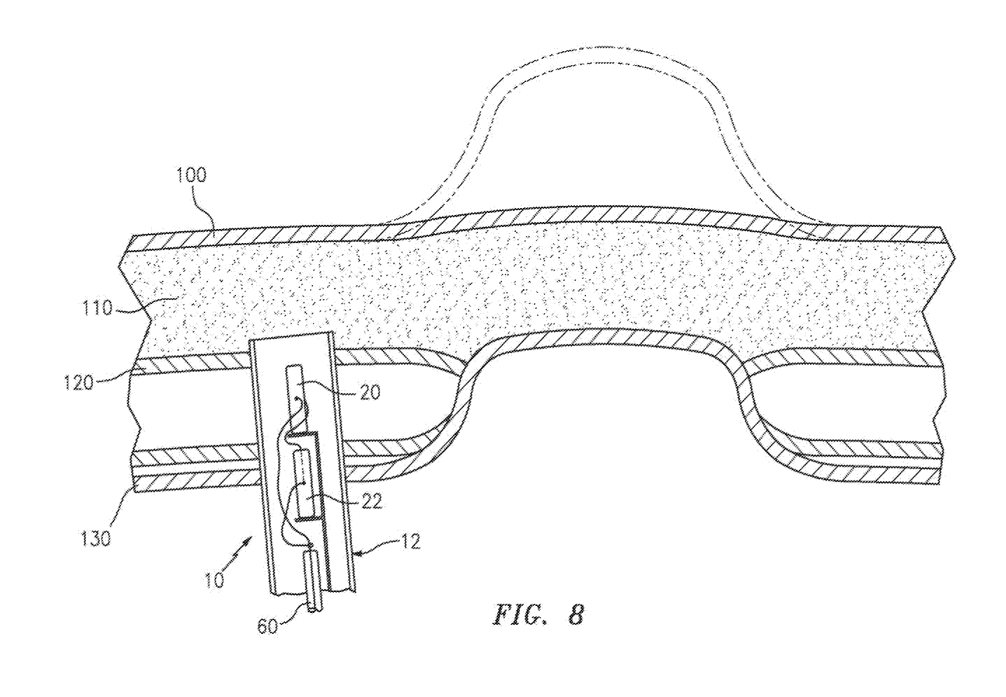

Turning now to the figures in connection with the aforementioned and below mentioned disclosure, it can be seen that FIGS. 1-7 illustrate preferred embodiments of the assembly for repairing a hernia, while FIGS. 8-13 illustrate steps to carry out preferred methodologies of the present invention.

In view thereof, reference is first made to FIG. 1, which illustrates an assembly, generally indicated at 10, for the repair of a hernia, constructed in accordance with a first embodiment of the present invention. In accordance with this first embodiment, assembly 10 preferably comprises a hollow tube, generally indicated at 12. Tube 12 is preferably made of stainless steel, with a diameter, for example at a front end 12A, of approximately 2-3 mm.

As would be understood in the art, the circular top edge 12B of front end 12A of tube 12 may be used as the cutting edge for cutting through any soft tissue and the abdominal wall as contemplated herein.

Optionally, shaft 12 may be disposed within an outer elongated shaft, generally indicated at 14, which, in this first embodiment is preferably connected to tube section 12 by an annular connector 16 connecting the outer circumference of shaft 12 with the inner circumference of shaft 14. Connector 16 may be as simple as one or more connecting rods that are glued, coupled, integral with and/or otherwise adhered or connected between the inner surface of outer elongated shaft 14 and the outer surface of tube 12, and which assist in maintaining the connection between tube section 12 and outer elongated shaft 14.

As would thus also be understood in the art, the front end 14A of the shaft 14 may alternatively be used as the cutting edge for cutting through any soft tissue and the abdominal wall as contemplated herein. Therefore, a spring 15 may extend about the circumference of the wall of the shaft 14 as appropriate to ensure the proper pressure is applied in the various embodiments, and just as importantly, to reduce the risk of unintentional and/or undesirable cutting of internal organs or which may otherwise occur during the procedure being described herein. The construction and/or selection of a suitable spring member or assembly would be understood by those skilled in the art. At least the front end 14A of the outer elongated shaft 14 is preferably made of stainless steel, with the remainder being in plastic if preferred. It should be understood that any of the assemblies disclosed herein may incorporate an outer shaft 14 and the figures should be understood as such.

In this first embodiment, assembly 10 also comprises a first holding member 20, which is at least mostly positionable in the tube 12. Preferably, first holding member 20 is positionable completely in tube 12, but it is contemplated that a portion of first holding member 20 may extend outward from front end 12A of tube 12 as insertion into the abdominal wall is proceeding. For example, in such an embodiment, it might be the first holding member 20 that itself has a cutting edge for piercing through the peritoneum and fascia layers of the abdominal wall.

Assembly 10 also comprises a second holding member 22, which similarly and preferably completely positionable in tube 12. In the embodiment of FIG. 1, and prior to their urging or deployment out of tube 12 as will be discussed below, both the first and second holding members 20, 22 are completely within tube 12. The particular relative positioning of the first and second holding members 20, 22 in FIG. 1 is exemplary, as those skilled in the art could position holding members 20, 22 within the tube 12 differently from that illustrated herein while still remaining within the scope of the present invention. Preferably, centering the holding members 20, 22 in the shaft/tube 12 will permit the incorporation of a uniform annular premature-deployment arrangement 45 as disclosed more fully below. It should be understood that the figures and text herein support embodiments in which the holding members are centered with a uniform annular ring 45 and should the figures need to be modified as such, no new matter would be added thereby.

As also illustrated in FIG. 1, a coupler, generally indicated at 25, couples the first holding member 20 to the second holding member 22. In a preferred embodiment, coupler 25 is a suture as would b understood in the art, but it should be understood that other forms of couplers could be used, including, but not limited to, thread, string, wire, cable, or any other material that can carry out the functionality thereof as explained herein. As used herein, the term “coupler” may be a one-piece assembly (i.e. a suture) or may be comprised of various components that are otherwise coupled or connected to each other as would be understood in the art. In this preferred embodiment, coupler 25 includes a locking arrangement 25A and an intermediate portion 25B. Locking arrangement 25A may be a fastener, clip or other locking device such as a slip-knot. In a preferred embodiment, locking arrangement 25A is a self-tightening knot 25A, and reference thereto will be made hereinafter. For the ease of description, the entire length of coupler 25, other than the portion that comprises knot 25A will be deemed and construed as the intermediate portion 25B for ease of description for purposes of the description and claims.

It should be pointed out that there are several contemplated embodiments of the coupler 25 for coupling the first and second holding members 20, 22. For example, in the embodiment of FIG. 1, self-tightening knot 25A extends out of a through-hole 23 in second holding member 22, with the intermediate portion 25B of coupler 25 extending through the rear of through-hole 23 and then through a through-hole 21 in first holding member 20 as illustrated in FIG. 1. In this way, and as discussed below, a pulling on the intermediate portion 25B, for example, from an end region, generally indicated at 25C of intermediate portion 25B, will cause the knot 25A to self-tighten and lock upon intermediate portion 25B. Through-hole 23 is sized to prevent knot 25A from receding therethrough. To be sure, there is no particular specific region that must be designated “end region” 25C. Rather, it is only for convenience that it should be understood that pulling on any section of coupler 25 extended out of the through-holes will be understood to be the “intermediate portion” and carry out the function of tightening the knot 25A as discussed herein. This concept should be understood in to be applicable in all embodiments.

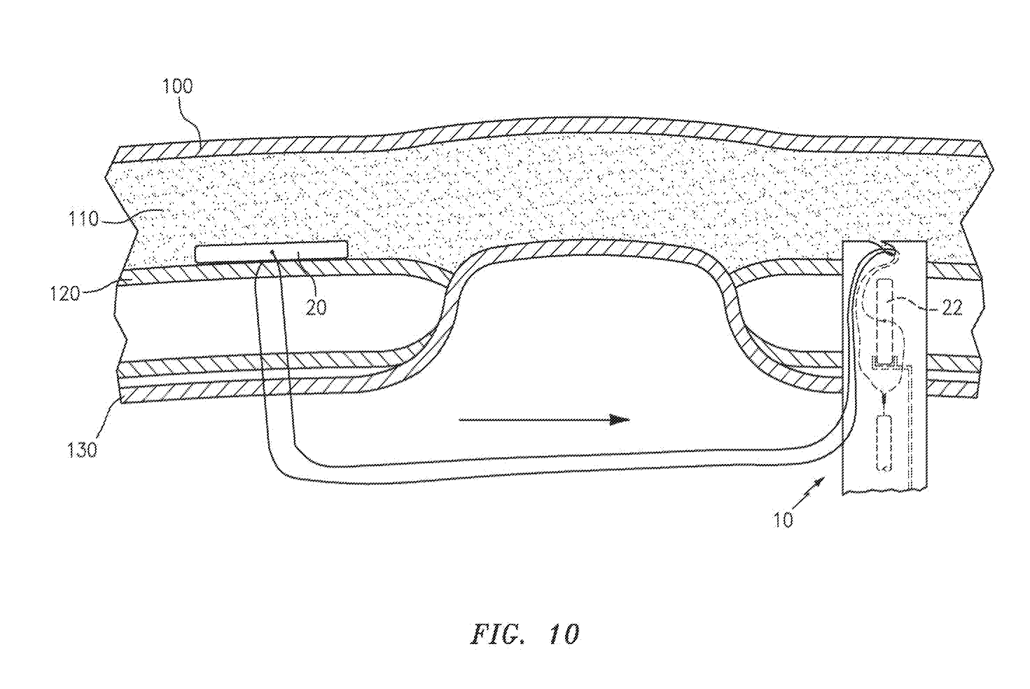

Assembly 10 also comprises a deployment assembly, generally indicated at 30, for deploying the first holding member 20 out the front end 12A of the tube 12 after the first end of the tube 12 has been extended and/or otherwise pierced through the peritoneum layer at a first position and through the fascia layer at a first position (FIG. 8 shows the tube inserted into the peritoneum and fascia layers at their respective first positions). The deployment assembly 30 also preferably functions to deploy the second holding member 22 after the first end of the tube 12 has pierced or otherwise extended through the peritoneum layer at a second position and through the fascia layer at a second position (FIG. 10 shows the tube inserted into the peritoneum and fascia layers at their respective second positions). Reference to the aforementioned “first positions” and “second positions” will be further discussed below. It should also be understood that some skilled in the art may move the peritoneum layer aside and therefore, piercing only of the fascia layer is needed. This step of moving aside the peritoneum layer is not material to the present invention. Moreover, reference to piercing of the peritoneum and fascia layers is made with respect to the first end 12A of tube 12. However, as alluded to above, it might be shaft 14 that does the piercing through the layers. Therefore, “piercing” of the layers by shaft 12 should also be understood throughout to mean simply “extending through” such layers.

In order to optimize the operability of the present invention and most importantly, to avoid tearing, cutting or otherwise comprising the integrity of coupler 25 as the tube 12 is moved in and out of the fascial/peritoneum layers at the first position and moved in/out of the fascial/peritoneum layers at the respective second positions, an opening or slot 13 (herein generically referenced by the term “opening” for convenience), may be provided below a top edge of the first end 12A of the tube 12. As will be further discussed below, a portion of the coupler 25 will be positioned in the opening 13 as the first end 12A of the tube 12 pierces through the peritoneum layer and the fascia layer at their respective first and second positions.

In addition, the coupler 25 must be restrained from being cut or otherwise compromised by contacting or otherwise engaging the top edge 12B of the first end 12A of the outer tube 12 as the first end 12A of the outer tube 12 pierces or otherwise extends through the peritoneum layer and the fascia layer at their respective first positions.

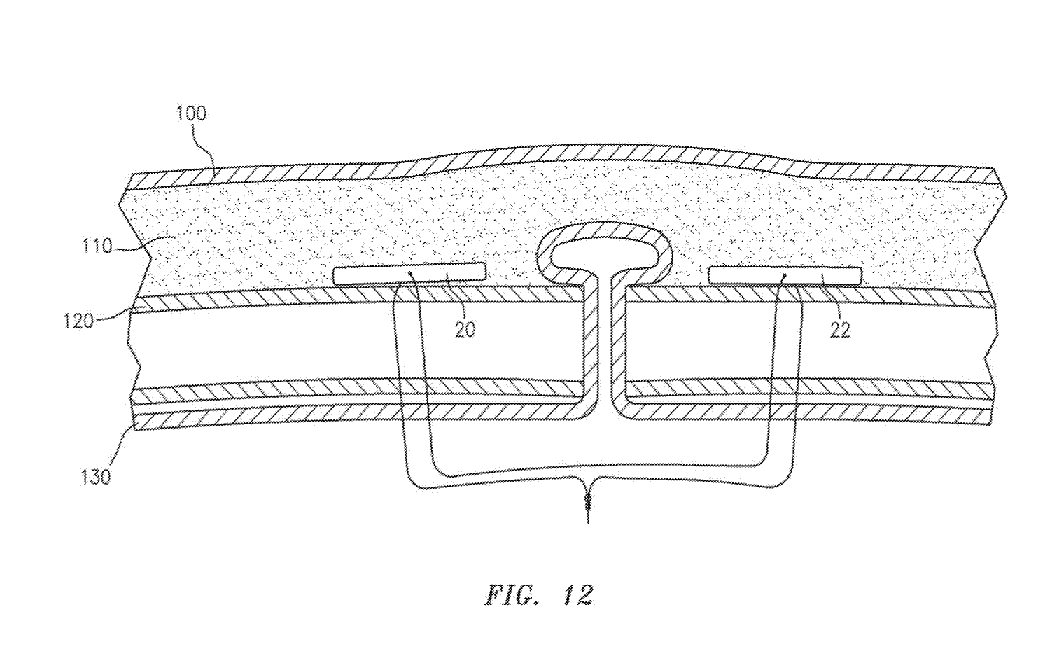

To this end, the assembly 10 also comprises means for restraining the coupler from being cut or otherwise compromised by engaging the top edge of the first end 12A of the outer tube 12 until after the first end of the elongated shaft pierces or otherwise extends through the peritoneum layer and the fascia layer at their respective first and second positions. The means, as illustrated in the figures, is made with reference to restraining member 50, which when in position, restrains the coupler 25 from exiting the opening 13 (and thus prevents it from being cut). However, when the member 50 is moved aside manually or by a separate device (not shown), coupler 25 is permitted to “escape” from the opening 13 and be free to exit out of the opening 13 and thus also out the first end of the tube 12, as illustrated for example in FIGS. 4 and 12, which thus facilities the full deployment of the second holding member 22 as well.

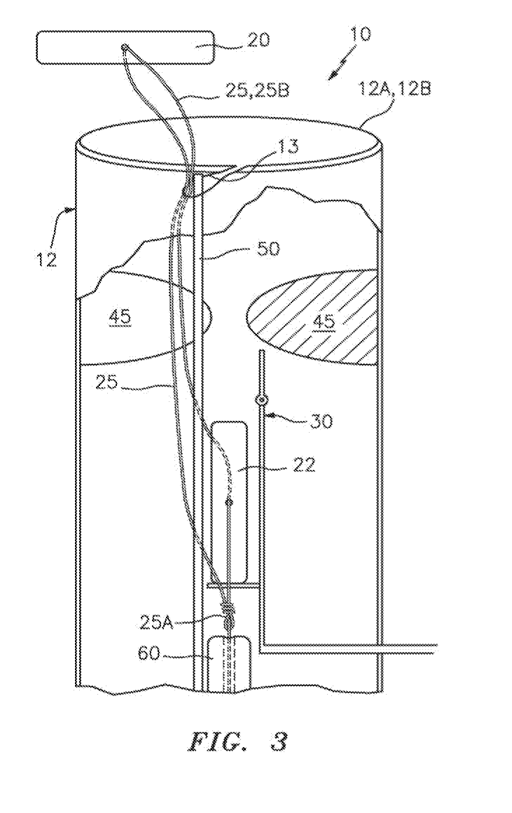

Restraining member 50 may be positioned in a variety of ways. For example, FIG. 1 illustrates a first end of the restraining member 50 placed across the opening 13. In FIG. 1, the restraining member may be held against the outer surface of tube 12. FIG. 2 alternatively illustrates restraining member 50 place across the opening 13 but held in place against the inner surface of tube 12. In either embodiment, the end of the restraining member 50 that was previously held across opening 13 is to be moved aside and/or away so that the coupler can escape from the opening 13, as illustrated in FIG. 4.

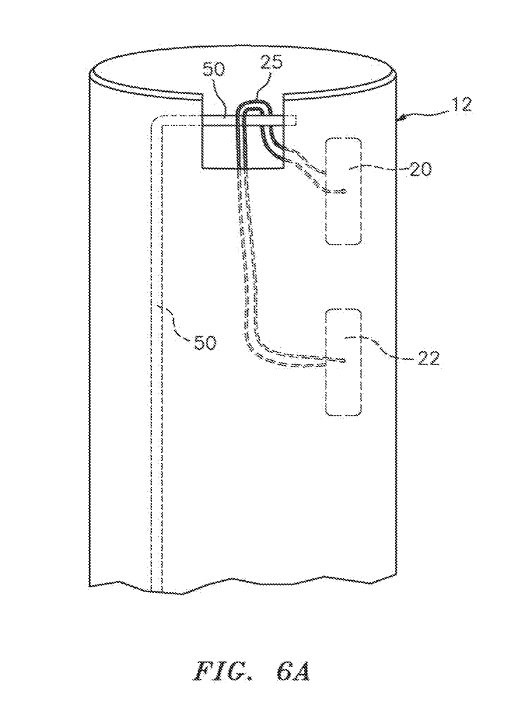

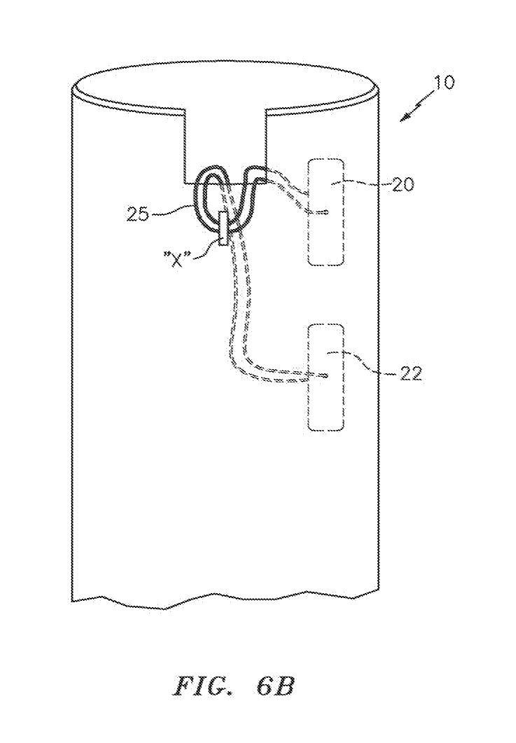

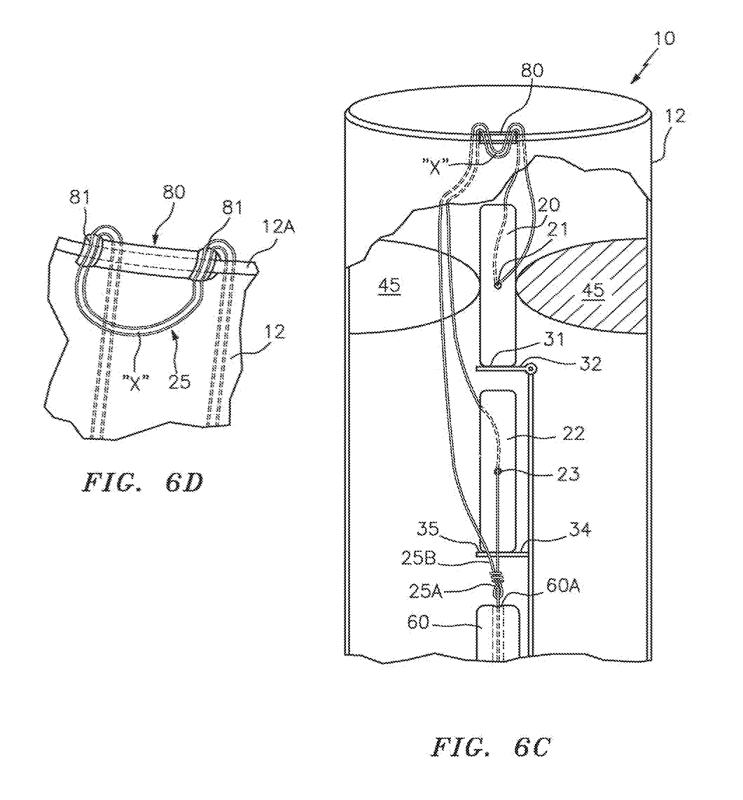

Alternatively, FIGS. 6A and 6B illustrate yet other preferred embodiments in which in FIG. 6A the opening 13 may be vertically oriented and the top edge need not be offset as illustrated in FIG. 1. This alternative embodiment, however, is similar in many respects to the embodiment of FIG. 1 in that importantly, the opening permits the coupler to remain below the top edge of the elongated shaft as the first end of the elongated shaft 12 pierces or otherwise extends through the peritoneum layer and the fascia layer at their respective first positions. In this alternative embodiment, once the first holding rod 20 is deployed and after the first end of the tube 12 has pierced or otherwise extended through the peritoneum layer at its second position and through the fascia layer at its second position, the restraining member 50 is moved or otherwise urged out of the way so that the second holding member can be deployed as disclosed herein. In yet an alternative embodiment, the end of the restraining member 50 that is positioned across the opening 13 may be provided as being sufficiently flexible such that a simple “tug” on the coupler will allow the end of the restraining member 50 to simply bend or otherwise “flex” out of the way. This might be preferred instead of physically moving the member 50 away from the opening.

Reference is next made to FIG. 6B, which illustrates yet another preferred embodiment in which there need not be a gate or physical structure to restrain the coupler as set forth above. Rather, the restraining means/member 50 may also be adhesive, glue, tape or the like that couples the coupler 25 to the outside surface of tube 12, for example in the square region indicated by the arrow X. In this manner, opening 13 may likewise be, but not necessarily, vertically oriented and the top edge need not be offset as illustrated in FIG. 1. This alternative embodiment, however, is similar in may respects to the embodiment of FIG. 1 in that importantly, the opening 13 permits the coupler 25 to remain below the top edge of the elongated shaft 12 during both piercings as disclosed herein. Here, once the first holding member is deployed and after the first end of the elongated shaft 12 has pierced or otherwise extended through the peritoneum layer at the second position and through the fascia layer at the second position, a gently pull or tug on the coupler 25 will be sufficient to decouple the coupler 25 from the surface of the tube 12 so that the second holding member 22 can be deployed as set forth above.

As noted above, several of the preferred embodiments envision the incorporation of an opening below the top edge of the first end 12A within which a portion of the coupler 25 is maintainable prior to the deployment of the second holding member 22. And, with such embodiment, the restraining member 50 restrains the portion of the coupler in the opening whereby the portion of the coupler in the opening is prevented from contacting the top edge of the first end of the elongated shaft 12 while the first and second holding members are positioned in the elongated shaft.

However, alternative embodiments are further envisioned. For example, FIG. 6C illustrates the use of a protective member 80 positioned on the top edge of the first end 12A of the elongated shaft 12 for protecting the coupler 25 from contacting the top edge of the first end of the elongated shaft 12 while the first and second holding members are in the elongated shaft (i.e. before deployment of both the first and second holding members 20, 22). Protective member 80 may be a cover or sheath, made from plastic, metal or otherwise, having a smooth surface and/or “channels” 81, 81 for further maintaining the position of the portion of the coupler 25 that extends over and onto the top edge of the shaft 12 (and ensuring that the coupler hanging across the top edge of tube 12 does not slip off and get cut or otherwise compromised by the top edge of the tube 12). FIG. 6D is an enlarged illustration of the protective member 80 of FIG. 6C. In FIG. 6D, the restraining means/member 50 may again be adhesive, glue, tape or the like that couples the coupler 25 to the outside surface of tube 12, for example in the square region indicated by the arrow X, being disengaged from the surface of the tube 12 in a similar manner as disclosed above with respect to the other embodiments.

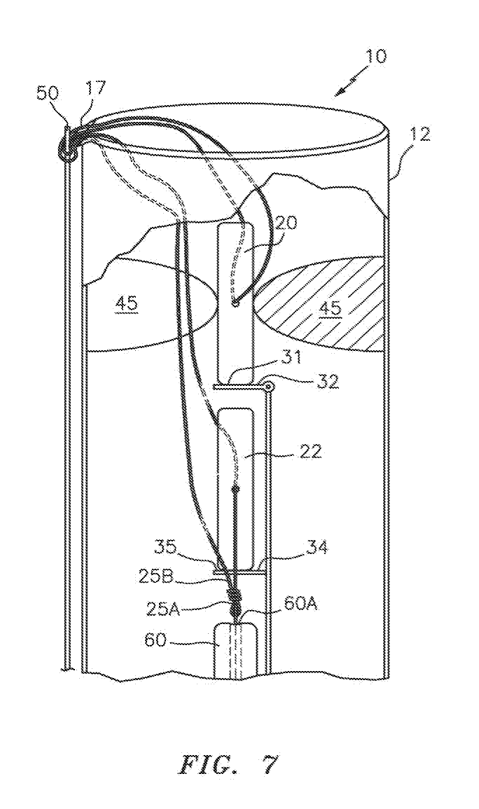

Still further and as illustrated in FIG. 7, instead (or in lieu) of an opening below the top edge 12B of first end 12A of shaft 12, a notch or slot (generically indicated as “notch” 17) may be provided in the top edge of first end 12A of shaft 12. Using such a notch, it would then be preferable to use one or more of the restraining members 50 as contemplated herein to maintain the coupler within the notch 17 so as not to be cut or otherwise compromised by the top edge of the shaft 12 until the second holding member is deployed as otherwise disclosed herein. In FIG. 7, it is shown that the restraining member is on the outside of the shaft 12. Alternatively, the restraining member could be held across the notch 17 from the inside surface of shaft 12. Still further alternatively, the coupler could be within the notch 17 and held using adhesive, glue, tape or the like against the outer surface as shown in FIGS. 6B or 6C where the X is shown to indicate the glue, tape or other adhesive material.

As noted above, for the convenience of the reader, the scale of the features as disclosed herein are not accurately depicted in the figures, although those skilled in the art will most clearly understand the proper scale and relative sizes of such features. However, importantly to mention is the desirable and preferable feature of a premature-deployment arrangement, generally indicated at 45 (see FIGS. 1, 5A) for preventing at least one of the first and second holding members 20, 22 from prematurely deploying from the first end of the elongated shaft 12. Preferably, the premature-deployment arrangement 45 comprises at least one protrusion (and at least preferably two) or an annular ring (also noted with reference number 45) on an inside wall surface of the elongated shaft 12, which remains in pressing contact with at least one of the first and second holding members 20, 22. Alternatively, deployment arrangement 45 may have a gap or slot therein to accommodate the coupler 25 that must pass thereby. Such a slot is implied by the depiction in FIGS. 1 and 5A. Still further alternatively, if arrangement 45 is a complete ring, then it would be understood that coupler 25 will be squeezed in with holding member 20 in the space within the ring of arrangement 45 (i.e. in the “donut hole”) where holding rod 20 is positioned in FIG. 1. In other words coupler 25 will be held between the surface of ring 45 and the outer surface of member 20. In a similar way, restrainer 50 will preferably also extend within the interior of ring 50 or may, as disclosed herein, be placed on the outside surface of tube 12.

For example, as illustrated in FIGS. 1, 5A, there is a protrusion on each side of (but more exemplary, an annular ring around) holding member 20. This/these protrusions or ring, if desired, could extend around the inner surface of shaft 12. In this way, holding members 20, 22 are frictionally prevented from premature deployment (i.e. they cannot simply fall out of the shaft 12). It should be understood that arrangement 45, whether as one or more protrusions (e.g. bumps) on the inner surface of shaft 12 or as an inner ring that extends around the inner surface of shaft 12, is envisioned to be on the inner surface of each and every embodiment disclosed herein. Protrusions or ring 45 may be made from flexible and/or compressible materials as would be understood in the art. Thus, although this feature of the premature-deployment arrangement 45 is not illustrated in each of the figures, it should be envisioned to be included therein and should be understood as such.

For the avoidance of doubt, FIG. 1 has the premature-deployment arrangement 45 illustrated therein, although, because of the illustrations of the other features of this embodiment, is not shown to be symmetrical about the figure. That is, in a preferred embodiment, holding members 20, 22 may be centered within the shaft 12 thereby accommodating and working well with a uniform ring or protrusions 45 about the inner surface of the shaft to symmetrically and frictionally retain holding members 20, 22 in the shaft. Should there only be one ring 45 for example, it would be understood that after holding member 20 is deployed, the position of holding member 22 will be such that it then would be engaging ring 45 until it too is urged out of the shaft by deployment assembly 30.

Once holding members 20, 22 are properly deployed as will be disclosed more fully below, a tension on the coupler 25 (e.g. by pulling at end 25C) pulls the first holding member 20 and the second holding member 22 towards each other, and further results in the locking arrangement 25A locking about the coupler 25. As will be understood more fully with respect to the figures discussed below, a defect in the fascia layer is closed thereby (see FIG. 12).

In a preferred embodiment, deployment assembly 30 comprises a pusher that may comprise a first urger 32 having an“urging” surface 31 to engage and deploy an end of first holding member 20 out of the front end 12A of tube 12. If desired, first urger 32 may be “cup” or “U” shaped to further ensure the proper engaging and urging of member 20 so it does not slide off or away from the urging surface 31. In a preferred embodiment, first urger 32 may include a hinge 33 that opens when pulled back into tube 12 after the first holding member 20 has been deployed, so that the first urger 32 does interfere or otherwise impede the deployment of second holding member 22 as will be disclosed below.

Deployment assembly 30 also preferably comprises a second urger 34 having an “urging” surface 35 to engage and deploy an end of second holding member 22 out of the front end 12A of tube 12. Likewise, if desired, urger 34 may be “cup” or “U” shaped to further ensure the proper support and engaging of member 22 so it does not slide off or away from the urging surface 35. Urger 34 need not include a hinge 33.

FIGS. 5A-D show a plurality of yet alternative deployment assemblies for deploying the first and/or second holding members. For example, the deployment assembly 30 may comprise: two separate pushers (FIG. 5A); or single pusher and a separate hinge device for retaining the upper holding member 20 in position until ready for deployment, whereby the single pusher sequentially deploys each holding rod (FIG. 5B); or two separate pushers with a hinge mechanism for retaining the upper holding member 20 in position prior to deployment (FIG. 5C); and/or two separate pushers wherein one of the pusher is inserted from a side port in tube 12 (FIG. 5D), and/or any combination of the foregoing. And, yet another deployment assembly provides that, with reference to FIG. 5A, holding members 20, 22 may simply be “stacked” on top of each other, wherein the pushing of deployment assembly 30 from under holding member 22 will urge holding member 20, being under frictional restraint from arrangement 45, out of the shaft 12. Thereafter, further urging of the second holding member 22 from assembly 30A from the bottom will thereby urge member 22 out of the shaft 12, thus making member 30B unnecessary in this alternative embodiment. In yet another alternative embodiment, the deployment assembly 30 may comprise the inner shaft 60 itself, thereby making a separate assembly 30 unnecessary. This would be possible with the holding members 20, 22 axially aligned as in FIGS. 1 and 5A.

As it relates to all the embodiments and figures herein, it should be understood than a separate premature-deployment arrangement 45 may be associated with holding member 22 or, as mentioned above, after holding member 20 is deployed, the position of holding member 22 will then be that previously occupied by holding member 20 thereby engaging arrangement 45. Also, it should be understood that arrangement 45 will be pliable and soft enough to permit the deployment assembly to pass thereby as needed. Alternatively, as disclosed herein, the inner shaft 60 can be used as the deployment assembly, which might further avoid any deformation of arrangement 45.

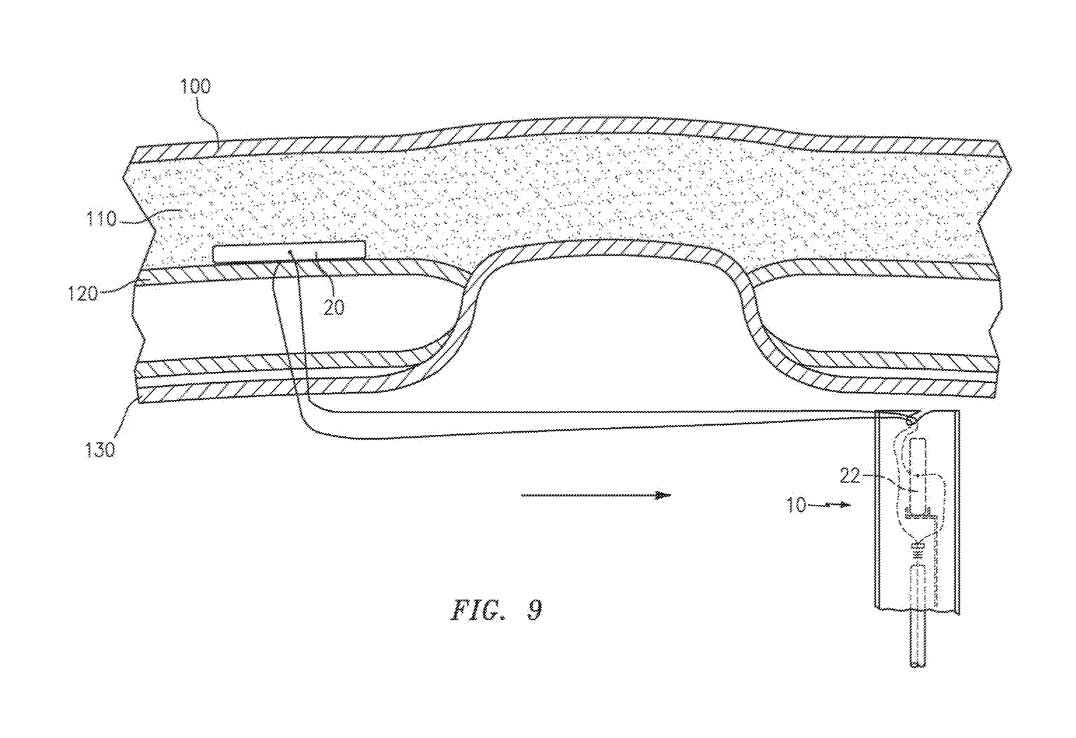

FIG. 2 illustrates assembly 10 after the first holding member 20 has been deployed, and as illustrated in FIG. 9, into the fatty layer of the abdominal wall after the first end 12A of the outer tube 12 has pierced or otherwise extended through the peritoneum layer at a first position and through the fascia layer at a first position. As illustrated in FIG. 2, the coupler 25 is still positioned in the opening 13, and importantly still positioned below the top edge of the first end 12A of the outer tube 12. In this way, the coupler 25 will not be cut or otherwise compromised as the first end 12A of the outer tube 12 is next moved into position and pierces through the peritoneum and fascia layers at the respective second positions (e.g. see FIGS. 9, 10).

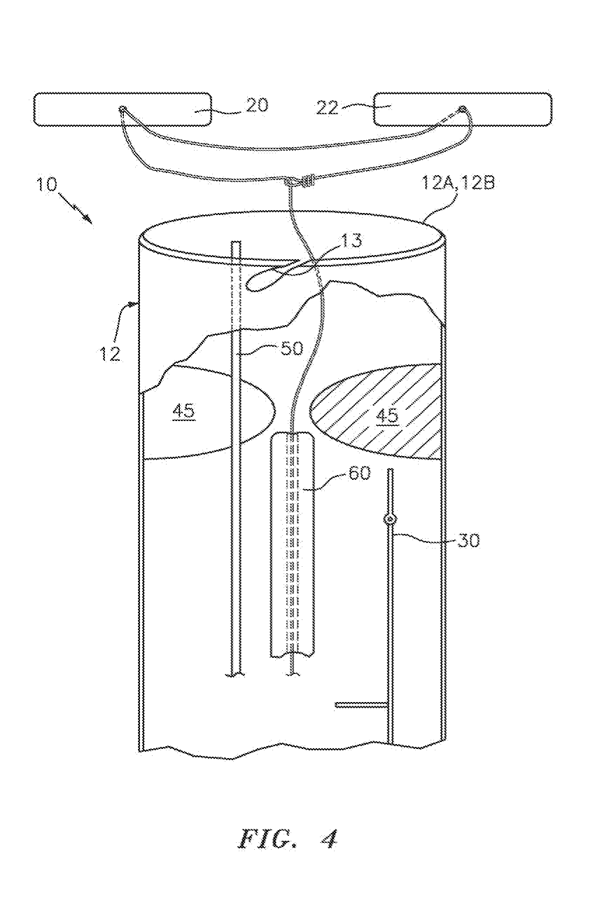

FIG. 3 illustrates a step after deployment of first holding member 20, but shows the coupler still remains restrained by the restraining member 50 away from the opening 13. The function of any of the restraining members should be likewise understood with respect to any of the other embodiments (e.g. the notch of FIG. 7, the openings of FIG. 6A or 6B or the embodiment of FIGS. 6C/6D, i.e. restraining the coupler prior to the deployment of the second holding member 22. On the other hand, FIG. 4 illustrates a step after deployment of second holding member 22, namely the movement of the restraining member 50 (i.e. away from the opening 13, notch 17 or separating the coupler from the shaft 12, for example), which would then permit the coupler from “escaping” from opening, notch or otherwise shaft generally, and facilitate the deployment of second holding member 22.

Reference is now made to FIGS. 8-12 for an understanding of preferred embodiments of the present invention using assembly 10 as disclosed herein. It should be also understood that the following methodologies are applicable to each and all of the embodiments set forth above regarding the construction of various assemblies and this disclosure should be considered as disclosing use of all the embodiments in connection with the following methodology.

FIG. 8 illustrates assembly 10 having been inserted into the peritoneum layer and fascia layers at their respective first positions. For example, as positioned, first holding member 20 can be freely deployed out of tube 12 and movable in the region intermediate the fascia layer 120 and the skin layer 100.

The deploying of first holding member 20 out the distal end 12A of the tube 12 can be achieved using one of the deployment assemblies disclosed herein. If the holding member 20 does not quickly and independently lie flush against the fascia layer as illustrated in FIG. 9 after deployment, a simple jostling or toggling of the coupler may be needed to ensure that first holding member 20 moves into flush engagement against the fascia layer.

Next, assembly 10 and tube 12 will withdrawn out of the abdominal wall and moved to the position also generally indicated in FIG. 9, where it will then be desirable to insert second holding member 22.

Assembly 10 is next inserted into the peritoneum layer and fascia layers at their respective second positions as illustrated in FIG. 10. Thereafter, second holding member 22 is to be deployed into the region intermediate the fascia later 120 and the skin layer 100.

Specifically, the exiting of second holding member 22 out the distal end 12A of the tube 12 can also be achieved in a variety of ways. For example, second holding member 22 can be pushed out the distal end 12A of the tube 12 using one of the deployment assemblies disclosed herein, or it can be pulled passively by the knot 25A of coupler 25. Using any of these two preferred ways or any other way consistent with the present invention, second holding member 22 will be jostled if needed to also lie flush against the fascia layer. To be sure, deployment assembly 30 can be, but need not be, used to deploy second holding member 22 and this applies to the deployment of all second holding members disclosed herein. To be sure, deployment member 30 is depicted as a pusher designed to be integrated into assembly 10. However, it is contemplated that deployment member 30 in all embodiments may similarly be a pusher insertable from a rear end of tube 12, which can likewise achieve its functionality as disclosed herein.

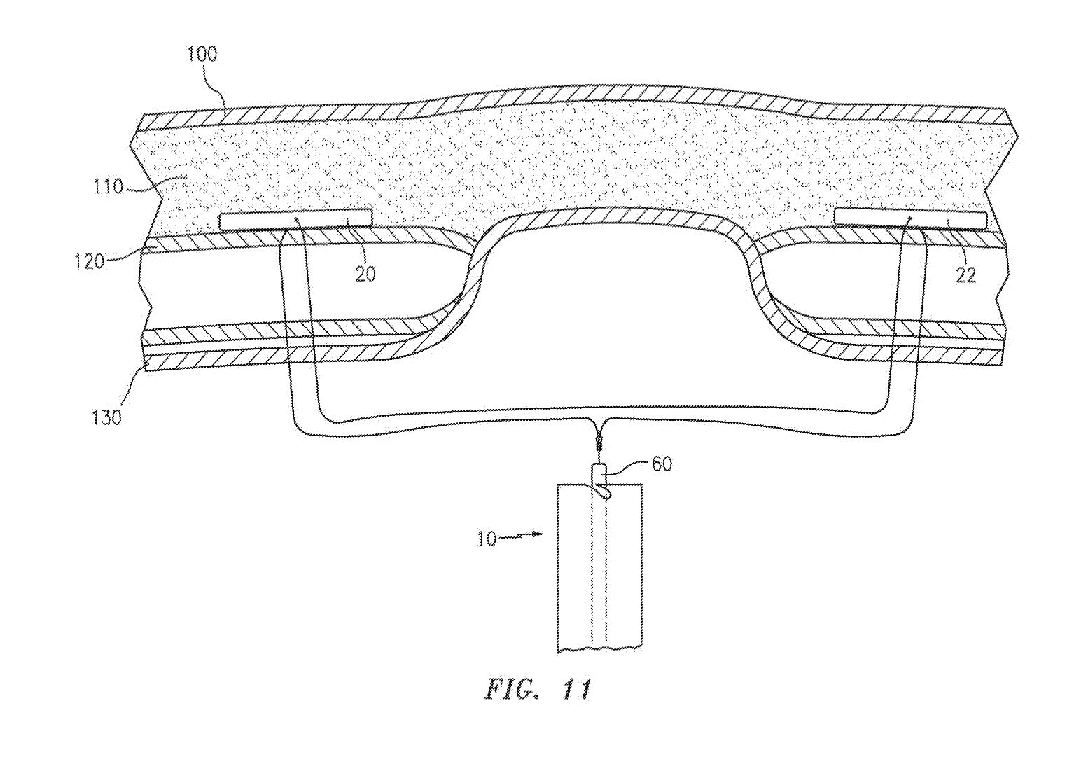

FIG. 11 illustrates second holding member 22 after having been exited from tube 12 in a manner similar to the deployment of first holding member 20.

An inner elongated shaft 60 may also be provided in tube 12. Inner elongated shaft 60 may be positionable by sliding into tube 12 via the rear end thereof. Preferably, at least a section of intermediate portion 25B of the coupler 25 extends within inner shaft 60 as illustrated in FIG. 1. The front end 60A of shaft 60 has an opening sufficient to allow intermediate portion 25B to be pulled therethrough yet small enough to preferably prevent knot 25A from falling therein. In this way, the tip of inner elongated shaft 60 can facilitate in maintaining the position of the knot 25A as desired and permits the tightening of the self-tightening knot 25A by pushing on knot 25A as the intermediate portion 25B of the coupler 25 is pulled out and from the rear end of the inner elongated shaft 40. Alternatively (or in addition), inner elongated shaft 60 may include an opening or slot (not shown) in a side surface thereof out of which the intermediate portion 25B of the coupler 25 is pullable so as to apply tension on the intermediate portion of the coupler so as to cause the first holding member 20 to be pulled towards the second holding member, as discussed below. While both alternatives are adequate, portion 25B will either extend out the rear end or side surface of shaft 40, but not simultaneously.

As can thus be seen, the means for engaging the locking arrangement 25A while tension is applied to the intermediate portion 25B of the coupler 25 is preferably the inner elongated shaft 60, as the inner elongated shaft 60 facilitates in maintaining the position of the locking arrangement 25A as tension is applied to the intermediate portion 25B of the coupler 25. Alternatively, tube 12 may comprise a slot (not shown) in front end 12A thereof, wherein the slot maintains the position of the locking arrangement 25A as tension is applied to the intermediate portion 25B of the coupler 25.

A tension on the intermediate portion 25B of the coupler 25 (e.g. by pulling on a section of the intermediate portion 25B that extends out of the rear end or opening of shaft 60) causes the first holding member 20 and the second holding member 22 to be pulled towards each other and the tightening of the self-tightening knot 25A about the intermediate portion 25B of the coupler 25. In this way, holding members 20, 22 remain in locked engagement with each other, each of which are retained against the fascia layer 120 of the abdominal wall.

Applying a tension on the coupler pulls the first holding member and the second holding member towards each other, and which may thus also cause the fascia layer at the first position to be pulled towards the fascia layer at the second position, (see FIG. 12). The locking arrangement is thereafter lockable about the coupler, also illustrated in FIG. 12. In this way, the defect in the fascia layer is closed thereby as illustrated in FIG. 12.

It should be pointed out that there are several contemplated embodiments of the coupler for coupling the first and second holding members. For example, a self-tightening knot may extend out of a through-hole in second holding member. In this way, pulling on the coupler from an end thereof will cause the knot to self-tighten and lock upon the coupler. Other locking arrangements and coupling arrangements are illustrated and disclosed and discussed in the aforementioned PCT/US2014/038076, and therefore are incorporated herein as they are applicable to the present invention.

As stated above, the deployment assemblies may be pushers that may comprise a first end having a “cup” or “U” shape so as to engage and deploy an end of the holding members out of the front end of the shaft, again, as also disclosed in the aforementioned PCT/US2014/038076.

Unless otherwise specified, the materials from which the aforementioned assemblies are constructed would be well understood in the art. For example, the tube 12 and shaft 60 are preferably made from stainless steel or plastic as would be understood by those skilled in the art. The first and second holding members are preferably of stainless steel or plastic or absorbable or non-absorbable material as would be understood in the art. The deployment assembly may also be preferably made from plastic as would also be understood in the art.

The foregoing assemblies can thus be used in connection with the preferred methodologies in accordance with the present invention. For example, and generally speaking, the assemblies disclosed herein are designed to be able to be placed thru a trocar and approach from the intraabdominal side of the fascia.

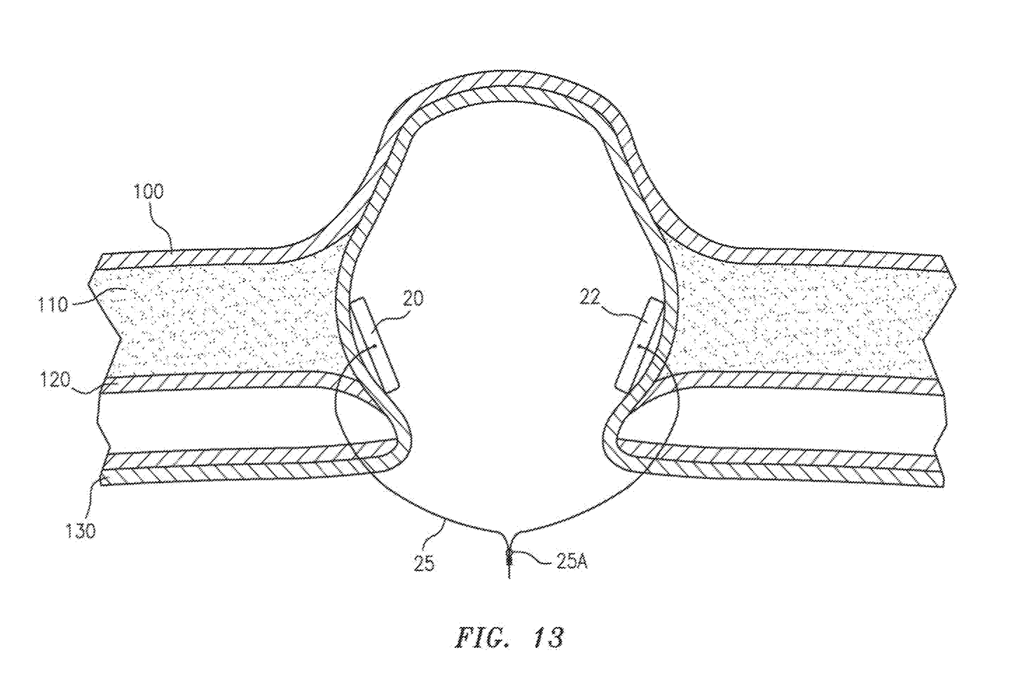

To be sure, each of the aforementioned steps are also applicable for carrying out the preferred methodology to repair the defect as illustrated in FIG. 13. Thus, one skilled in the art would understand how to apply the disclosure above to carry out the sequence of steps to achieve the result shown in FIG. 13, especially in view of the similar method as illustrated in FIG. 8-12.

Once the two holding members are pulled towards each other the locking arrangement (e.g. sliding loop knot closes (if a self-tightening knot is used)) fixates the two holding members in position. As a final step the suture is pulled and then cut by a scissors or other cutting edge. The cutting of the suture as a final step once the holding members are in position is applicable to all of the embodiments disclosed herein.

In addition, it is important to note that all of the foregoing embodiments have been disclosed with the idea in mind that the person(s) performing the methodology disclosed above and using the devices disclosed herein would be doing so from entering the abdominal wall from the peritoneum side thereof. However, further modifications and alternatives are contemplated by the present invention and within the scope of the claims herein. for example, while the first and second holding members will still close the defect in the fascia layer as contemplated herein, such may be accomplished by a further alternative method in which the various assemblies of the present invention have been inserted in the direction opposite to that illustrated in the foregoing figures, namely, that the respective assemblies are first inserted through the fascia layer of the abdominal wall and thereafter (optionally) through the peritoneum layer. Therefore, generally speaking, in this alternative methodology, the defect is closed by the first holding member and second holding member being retained against the fascia layer of the abdominal wall. Those skilled in the art would readily understand from the foregoing disclosure how the same sequence of steps would be performed to achieve the desired result, albeit from the direction opposite to that disclosed above. And still further, it is contemplated that the person(s) performing the above procedures may opt to move away or otherwise not pierce through the peritoneum layers for reasons that would be understood by those skilled in the art. As the reader will herein note, the claims and the disclosure should also therefore be understood to cover embodiments in which the peritoneum layer is not pierced. It can thus be seen that the present invention provides improved methodologies and assemblies for securing repairing a hernia and overcomes existing problems in the art.

Those skilled in the art would understand how to utilize a clip or other fastener in place of the preferred knot assembly.

As would thereafter be understood, the coupler 25 may be cut to remove the unneeded excess thereof.

It can thus be seen that the present invention provides improved methodologies and assemblies for repairing a hernia and overcomes existing problems in the art. For example, the present invention avoids unnecessary skin incisions. Furthermore, only basic laparoscopic skills are needed with the present invention. Still further, a reduction in operating time is realized by the use of the present invention. In addition, the use of the term “self-tightening” knot is intended to encompass all such knots that functionally carry out the objective of the invention, and therefore, are intended to include, without limitation, a locking knot, a slip knot and/or any other form of self-tightening knot as known or would be understood in the art.

In summary, among other things, the present invention allows the surgeon to close the abdominal wall defect with tremendous time savings during the surgery and it reduces the risk of a hernia recurrence.

To be sure, the present application incorporates by reference the subject matter of copending international application No. PCT/US2014/038076 as if set forth in its entirety.

The phrase “at least mostly” is intended to mean at least 80%.

Moreover, while the preferred embodiments and the claims are disclosed in a sequence as set forth herein, the claims are not limited thereby unless specifically recited as such. That is, the steps of the claims may be performed in a different order unless explicitly recited in a specific order.

Again, for completeness, there can be an opening, slot or gap in the ring 45 to permit the positioning of the restraining member 50 and coupler 25 in the shaft. Without such a ring, it would be understood that member 50 and coupler 25 will move much more freely within the inner cavity of shaft 12. One skilled in the art would understand the benefits and disadvantages of using a complete ring 45. In such an alternative, as disclosed herein, simple protrusions (e.g. 2, 4, 6 or 8 of them) may be used and spaced around the inner circumference to achieve the functional advantages set forth herein.

Also, terms that reference features herein may be viewed as being functionally defined (e.g. “urger,” “restrainer,” “premature deployment arrangement”), but this is for convenience only. That is, all of the features herein may alternatively defined simply by the term “member” or the like. Therefore, nothing should be deemed limiting by the use of the terms above, other than for convenience of the reader and to help define the functions and features afforded thereby.

In addition, it is noted that FIGS. 1-13 utilize a loop arrangement for coupler 25, which facilitates the use of a self-tightening slip knot to pull the holding members closer together as shown in FIG. 12. However, and as noted in FIG. 13, a different connection using coupler 25 can be used, namely one that does not utilize a loop as in the other figures. That is, although FIG. 13 can still use a self-tightening slip knot, it would have to be configured a bit differently. Thus, it should be understood that the coupler configuration in FIG. 13 can be used in FIGS. 1-12 and likewise, the looping coupler configuration in FIGS. 1-12 can be used in FIG. 13. That is, one skilled in the art would appreciate that different loop and connection configurations can be used for coupler 25. Moreover, some configurations (e.g. a loop) will lend themselves more easily to the use of a slip knot whereas others may not but the person performing the procedure may opt/choose to use a different type of locking arrangement (e.g. clip, clamp, etc). Thus, the figures should not be limited thereby.

It is also to be understood that the following claims are intended to cover all of the generic and specific features of the invention described herein and all statements of the scope of the invention which as a matter of language might fall therebetween.

1. an assembly for the repair of a hernia, comprising:
an elongated shaft having a first end;
a first holding member at least mostly positionable in the elongated shaft and a second holding member positionable in the elongated shaft;
a coupler assembly comprising (i) a coupler for coupling the first holding member to the second holding member and (ii) a locking arrangement;
a deployment assembly for deploying at least one of the first and second holding members out of the elongated shaft;
a restrainer for restraining a portion of the coupler prior to the deployment of the second holding member out of the first end of the elongated shaft;
wherein the coupler can be tensioned so as to pull the first holding member and the second holding member towards each other and the locking arrangement is lockable about the coupler.

2. The assembly as claimed in claim 1, wherein the first holding member is positioned fully in the elongated shaft.

3. The assembly as claimed in claim 1, wherein the elongated shaft comprises an opening below a top edge of the first end thereof within which a portion of the coupler is maintainable, and the restrainer restrains the portion of the coupler in the opening;
whereby the portion of the coupler in the opening is prevented from contacting the top edge of the first end of the elongated shaft while the first and second holding members are positioned in the elongated shaft.

4. the assembly as claimed in claim 1, wherein the elongated shaft comprises a notch in a top edge of the first end thereof within which a portion of the coupler is maintainable, and the restrainer restrains the portion of the coupler in the opening;
whereby the portion of the coupler in the notch is prevented from contacting the top edge of the first end of the elongated shaft while the first and second holding members are positioned in the elongated shaft.

5. The assembly as claimed in claim 1, comprising a protective member positioned on a top edge of the first end of the elongated shaft for protecting the coupler from contacting the top edge of the first end of the elongated shaft while the first and second holding members are positioned in the elongated shaft.

6. The assembly as claimed in claim 1, wherein the restrainer is selected from a group of restrainers comprising glue, adhesive or tape.

7. The assembly as claimed in claim 1, wherein the restrainer comprises an elongated member that is positionable across the opening and against at least one of the outer surface of the elongated shaft or against the inner surface of the elongated shaft.

8. The assembly as claimed in claim 1, comprising a premature-deployment arrangement for preventing at least one of the first and second holding members from prematurely deploying from the first end of the elongated shaft.

9. The assembly as claimed in claim 8, wherein the premature-deployment arrangement comprises (i) at least two protrusions, (ii) at least two protrusions and/or (iii) an annular ring on an inside wall surface of the elongated shaft, the premature-deployment arrangement in pressing contact with at least one of the first and second holding members.

10. The assembly as claimed in claim 1, comprising an inner elongated shaft for engaging the locking arrangement when tension is applied to an intermediate portion of the coupler, wherein the inner elongated shaft facilitates in maintaining the portion of the locking arrangement as tension is applied to the intermediate portion of the coupler.

11. The assembly as claimed in claim 10, wherein the deployment assembly is the inner elongated shaft.

12. The assembly as claimed in claim 1, wherein the locking arrangement is a self-tightening knot and wherein tension on the intermediate portion of the coupler causes the tightening of the self-tightening knot about the intermediate portion of the coupler.

13. The assembly as claimed in claim 10, wherein the inner elongated shaft comprises at least one of:
a rear end out of which the intermediate portion of the coupler is pullable so as to apply tension on the intermediate portion of the coupler so as to cause the first holding member to be pulled towards the second holding member; and
an opening in a side surface out of which the intermediate portion of the coupler is pullable so as to apply tension on the intermediate portion of the coupler so as to cause the first holding member to be pulled towards the second holding member.

14. A method of repairing a hernia by closing a hole in the fascia layer of an abdominal wall utilizing an assembly comprising an elongated shaft having a first end, a first holding member at least mostly positionable in the elongated shaft and a second holding member positionable in the elongated shaft, a coupler assembly comprising (i) a coupler for coupling the first holding member to the second holding member and (ii) a locking arrangement, a deployment assembly for deploying at least one of the first and second holding members out of the elongated shaft; a restrainer for restraining a portion of the coupler prior to the deployment of the second holding member out of the first end of the elongated shaft; wherein a tension on the coupler pulls the first holding member and the second holding member towards each other and wherein the locking arrangement is lockable about the coupler, wherein the method comprises the steps of:
extending the first end of the elongated shaft through at least a fascia layer of the abdominal wall at a first position;
deploying the first holding member out the first end of the elongated shaft;
removing the elongated shaft out of the abdominal wall; and
while the coupler is still coupled intermediate the first and second holding rods, extending the first end of the elongated shaft through at least the fascia layer at a second position;
deploying the second holding member out the first end of the elongated shaft;
removing the elongated shaft out of the abdominal wall and removing the restraint upon the coupler;
applying a tension on the coupler to cause the first holding member and the second holding member to be pulled towards each other;
locking the locking arrangement about the intermediate portion of the coupler,
whereby a defect in the fascia layer is closed thereby.

15. The method as claimed in claim 14, wherein the elongated shaft comprises an opening below a top edge of the first end thereof within which a portion of the coupler is maintainable, and the restrainer restrains the portion of the coupler in the opening; wherein the method comprises the step of:
restraining the portion of the coupler in the opening until the second holding member is deployed out of the first end of the elongated shaft.

16. The method as claimed in claim 14, wherein the elongated shaft comprises a notch in a top edge of the first end thereof within which a portion of the coupler is maintainable, and the restrainer restrains the portion of the coupler in the notch; wherein the method comprises the step of:
restraining the portion of the coupler in the notch until the second holding member is deployed out of the first end of the elongated shaft.

17. The method as claimed in claim 14, comprising the step of protecting the coupler from contacting the top edge of the first end of the elongated shaft while the first and second holding members are positioned in the elongated shaft.

18. The method as claimed in claim 14, comprising the step of restraining the coupler against either an outer surface of the elongated shaft or against the inner surface of the elongated shaft.

19. The method as claimed in claim 14, wherein the locking arrangement is a self-tightening knot, wherein the method comprises the step of applying the tension on the intermediate portion of the coupler causing the tightening of the self-tightening knot about the intermediate portion of the coupler.
